(19) United States
(12) Patent Application Publication
(10) Pub. No.: US 2026/0000408 A1
(43) Pub. Date: Jan. 1, 2026

(54) SURGICAL CLIP REQUIRING MINIMAL CLOSURE FORCE

(71) Applicant: Ethicon, Inc., Raritan, NJ (US)

(72) Inventors: Elizabeth A. Buck, Marlton, NJ (US); Robert A. Rousseau, Ottsville, PA (US); Everett A.H. Will, Cleveland, OH (US); Xuelin Guo, Livingston, NJ (US)

(21) Appl. No.: 18/757,997

(22) Filed: Jun. 28, 2024

(51) Int. Cl. A61B 17/122; A61B 17/128

(52) U.S. Cl. CPC A61B 17/122; A61B 17/1285; A61B 2017/00004

(57) ABSTRACT

A surgical ligation clip includes a first arm extending from a proximal end to a first distal end. The first arm includes a distal hook at the first distal end. The surgical ligation clip also includes a second arm extending from the proximal end to a second distal end. The second arm is pivotable relative to the first arm about a proximal hinge to clamp tissue between the first and second arms. The second arm includes a proximal portion and a distal portion. The distal portion is pivotable relative to the proximal portion about a distal hinge.

### BACKGROUND

Surgical clips may be used for a number of surgical procedures. In endoscopic or laparoscopic surgical procedures, access to the surgical site may be achieved through a trocar inserted through a small entrance incision in the skin. The trocar port allows the surgeon to insert a number of different surgical instruments therethrough and to perform surgical procedures within the patient in a minimally invasive manner.

During some surgical procedures, the surgeon may wish to terminate the flow of blood or another fluid through one or more vessels. In some such instances, the surgeon may apply a surgical clip to a blood vessel or another duct to prevent the flow of blood or other bodily fluids therethrough during the procedure. An endoscopic surgical clip applier is capable of applying a singular surgical clip or multiple surgical clips during a minimally invasive entry to the body cavity. For instance, an endoscopic surgical clip applier is capable of ligating a blood vessel by clamping a surgical clip about the blood vessel to thereby prevent blood flow through the vessel. Such clips may be fabricated from a malleable biocompatible material and may be compressed over a vessel. Alternatively, such clips may be fabricated from a resilient biocompatible material and may be released to resiliently clamp the vessel.

Examples of surgical clips are represented by the LIGACLIP® series of surgical clips by Ethicon Endo-Surgery, Inc. of Cincinnati, Ohio. One example of a surgical clip applier is the LIGAMAX™ 5 by Ethicon Endo-Surgery, Inc. of Cincinnati, Ohio. Other examples of surgical clip appliers are represented by the LIGACLIP® series of surgical clip appliers by Ethicon Endo-Surgery, Inc. of Cincinnati, Ohio. Still further examples of surgical clip appliers and surgical clips are disclosed in U.S. Pat. No. 5,163,945, entitled “Surgical Clip Applier,” issued Nov. 17, 1992, the disclosure of which is incorporated by reference herein; U.S. Pat. No. 5,342,373, entitled “Sterile Clips and Instrument for their Placement,” issued Aug. 30, 1994, the disclosure of which is incorporated by reference herein; U.S. Pat. No. 5,431,668, entitled “Ligating Clip Applier,” issued Jul. 11, 1995, the disclosure of which is incorporated by reference herein; U.S. Pat. No. 5,445,167, entitled “Methods of Applying Surgical Clips and Suture Tie Devices to Bodily Tissue During Endoscopic Procedures,” issued Aug. 29, 1995, the disclosure of which is incorporated by reference herein; U.S. Pat. No. 5,601,573, entitled “Sterile Occlusion Fasteners and Instruments and Methods for Their Placement,” issued Feb. 11, 1997, the disclosure of which is incorporated by reference herein; U.S. Pat. No. 5,951,574, entitled “Multiple Clip Applier Having a Split Feeding Mechanism,” issued Sep. 14, 1999, the disclosure of which is incorporated by reference herein; U.S. Pat. No. 7,261,724, entitled “Surgical Clip Advancement Mechanism,” issued Aug. 28, 2007, the disclosure of which is incorporated by reference herein; U.S. Pat. No. 7,686,820, entitled “Surgical Clip Applier Ratchet Mechanism,” issued Mar. 30, 2010, the disclosure of which is incorporated by reference herein; U.S. Pat. No. 7,699,860, entitled “Surgical Clip,” issued Apr. 20, 2010, the disclosure of which is incorporated by reference herein; U.S. Pat. No. 7,731,724, entitled “Surgical Clip Advancement and Alignment Mechanism,” issued Jun. 8, 2010, the disclosure of which is incorporated by reference herein; U.S. Pat. No. 8,038,686, entitled “Clip Applier Configured to Prevent Clip Fallout,” issued Oct. 18, 2011, the disclosure of which is incorporated by reference herein; and U.S. Pat. No. 8,262,679, entitled “Clip Advancer,” issued Sep. 11, 2012, the disclosure of which is incorporated by reference herein.

While various kinds of surgical clips and associated components have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1A depicts a side elevational view of an example of a ligation clip, showing the ligation clip in an open state;

FIG. 1B depicts a side elevational view of the ligation clip of FIG. 1A, showing an anatomical structure received between upper and lower arms of the ligation clip, and showing the ligation clip transitioned to a partially closed state by a robotic applicator;

FIG. 1C depicts a side elevational view of the ligation clip of FIG. 1A, showing the ligation clip transitioned to a fully closed state by the robotic applicator to ligate the anatomical structure;

FIG. 2 depicts a perspective view of another example of a ligation clip;

FIG. 3 depicts a partial perspective view of the ligation clip of FIG. 2;

FIG. 4A depicts a side elevational view of the ligation clip of FIG. 2, showing the ligation clip in an open state;

FIG. 4B depicts a side elevational view of the ligation clip of FIG. 2, showing the ligation clip in a closed state;

FIG. 5A depicts a side elevational view of another example of a ligation clip, showing the ligation clip in an open state;

FIG. 5B depicts a side elevational view of the ligation clip of FIG. 5A, showing the ligation clip in a partially closed state;

FIG. 5C depicts a side elevational view of the ligation clip of FIG. 5A, showing the ligation clip in a fully closed state;

FIG. 6A depicts a side elevational view of another example of a ligation clip, showing the ligation clip in an open state;

FIG. 6B depicts a side elevational view of the ligation clip of FIG. 6A, showing the ligation clip in a closed state;

FIG. 7 depicts a side elevational view of another example of a ligation clip;

FIG. 8 depicts a partial perspective view of the ligation clip of FIG. 7;

FIG. 9 depicts a bottom plan view of the ligation clip of FIG. 7;

FIG. 10A depicts a side elevational view of the ligation clip of FIG. 7, showing an anatomical structure received between upper and lower arms of the ligation clip, and showing the ligation clip in an open state;

FIG. 10B depicts a side elevational view of the ligation clip of FIG. 7, showing the ligation clip in a first partially closed state;

FIG. 10C depicts a side elevational view of the ligation clip of FIG. 7, showing the ligation clip in a second partially closed state;

FIG. 10D depicts a side elevational view of the ligation clip of FIG. 7, showing the ligation clip in a fully closed state;

FIG. 11A depicts a side elevational view of another example of a ligation clip, showing the ligation clip in an open state;

FIG. 11B depicts a side elevational view of the ligation clip of FIG. 11A, showing the ligation clip in a closed state;

FIG. 12A depicts a side elevational view of another example of a ligation clip, showing the ligation clip in an open state;

FIG. 12B depicts a side elevational view of the ligation clip of FIG. 12A, showing the ligation clip in a partially closed state; and

FIG. 12C depicts a side elevational view of the ligation clip of FIG. 12A, showing the ligation clip in a fully closed state.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

### I. Examples of Surgical Ligation Clips

In some procedures, it may be desirable to provide a surgical ligation clip that is configured to be placed into the body of a patient via a minimally invasive and/or robotic procedure. In addition, or alternatively, it may be desirable for such a surgical ligation clip to be capable of transitioning from an open state to a fully closed state via the application of a minimal closure force to one or more arms of the ligation clip, and/or for such a surgical ligation clip to have one or more engagement members configured to secure the surgical ligation clip in the fully closed state. Each of the examples of surgical ligation clips (110, 210, 310, 410, 510, 610, 710) described below may function in such a manner. It will be appreciated that each of the examples of surgical ligation clips (110, 210, 310, 410, 510, 610, 710) described below may comprise a biocompatible material, and may include bioabsorbable material or any other suitable nonabsorbable material(s) for implanting within the human body, and/or may be manufactured via injection molding, 3D printing, machining to form or any other suitable manufacturing process(es).

### A. First Example of Surgical Ligation Clip

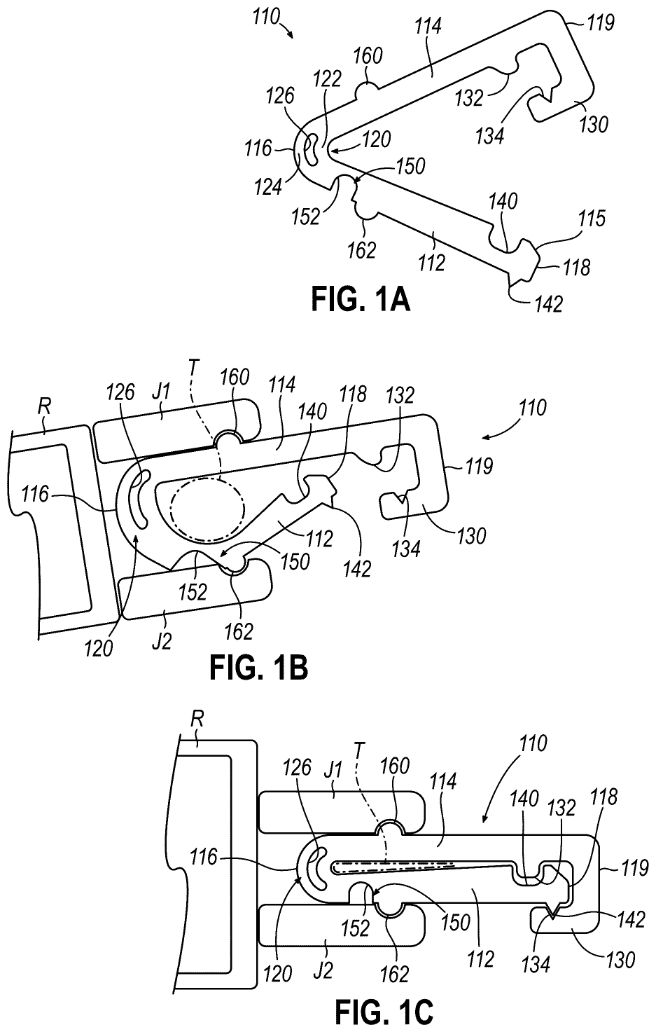

FIGS. 1A-1C show an example of a surgical ligation clip (110). As shown, ligation clip (110) of this example includes a pair of arms (112, 114) including a lower arm (112) and an upper arm (114) that are joined together at a proximal end (116). In the example shown, lower arm (112) extends from proximal end (116) to a lower distal end (118), and upper arm (114) extends from proximal end (116) to an upper distal end (119). A deformable proximal hinge element (120) is defined at or near proximal end (116) by an inner hinge wall element (122) and an outer hinge wall element (124) that are spaced apart from each other by a generally C-shaped slot (126), such that arms (112, 114) may be movable relative to each other about proximal hinge element (120). In some versions, inner hinge wall element (122) and outer hinge wall element (124) may have different thicknesses from each other. Such different thicknesses may be selected to minimize the magnitude of closure force required for bending ligation clip (110) about proximal hinge element (120).

Upper arm (114) of the present example includes a distal hook (130) provided at or near upper distal end (119). Distal hook (130) is sized and configured to securely receive lower distal end (118) of lower arm (112) when ligation clip (110) is in a fully closed state. In this regard, upper arm (114) of the present example also includes a pair of latching members in the form of a generally trapezoidal protrusion (132) extending downwardly from a lower surface of upper arm (114) slightly proximally of distal hook (130), and a generally triangular recess (134) extending downwardly from an upper surface of distal hook (130), which are each configured to interlock with corresponding mating features of lower arm (112) to maintain ligation clip (110) in the fully closed state once ligation clip (110) reaches the fully closed state. To that end, lower arm (112) of the present example includes a pair of latching members in the form of a generally rectangular recess (140) extending downwardly from an upper surface of lower arm (112) slightly proximally of lower distal end (118) and configured to receive protrusion (132), and a generally triangular protrusion (142) extending downwardly from a lower surface of lower arm (112) slightly proximally of lower distal end (118) and configured to be received by recess (134). In some versions, recess (140) and protrusion (132) may be sized and shaped relative to each other to provide a snap-fit therebetween. In addition, or alternatively, recess (134) and protrusion (142) may be sized and shaped relative to each other to provide a snap-fit therebetween.

In the example shown, a deformable distal hinge element (150) is defined distal of proximal hinge element (120) by a generally C-shaped recess (152) extending upwardly from the lower surface of lower arm (112), such that a distal portion of lower arm (112) may be movable relative to a proximal portion of lower arm (112) about distal hinge element (150). Distal hinge element (150) may thereby allow ligation clip (110) to be movable to a partially closed state as described in greater detail below.

As shown, an upper protrusion (160) extends upwardly from an upper surface of upper arm (114) and a lower protrusion (162) extends downwardly from the lower surface of lower arm (112) slightly distal of distal hinge element (150), which are each configured to be gripped by a corresponding jaw (J1, J2) of a robotic end effector in the form of a robotic applicator (R). As described in greater detail below, the robotic applicator (R) may transition ligation clip (110) between the open, partially closed, and fully closed states by applying one or more forces to protrusions (160, 162).

In this regard, ligation clip (110) may initially be in the open state, as shown in FIG. 1A. First and second jaws (J1, J2) of robotic applicator (R) may then grip the corresponding protrusions (160, 162) and maneuver ligation clip (110) to receive a tissue structure (e.g., vessel, etc.) (T) between arms (112, 114), and may further apply a closure force to ligation clip (110) via protrusions (160, 162) to transition ligation clip (110) to the partially closed state, as shown in FIG. 1B. More particularly, the urging of protrusions (160, 162) toward each other by jaws (J1, J2) may cause the distal portion of lower arm (112) to pivot upwardly about distal hinge element (150) relative to the proximal portion of lower arm (112), such that lower distal end (118) of lower arm (112) may be brought proximal of distal hook (130) without colliding with distal hook (130). Additionally, distal hinge element (150) may provide a lower resistance to applied bending forces than proximal hinge element (120). The differential in bending resistance forces may facilitate inward bending of distal end (118) towards upper arm (114) prior to deformation of the proximal portion of lower arm (112) that extends between proximal hinge element (120) and distal hinge element (150). The avoidance of a collision of the distal portion of lower arm (112) with distal hook (130) may ensure that the tissue structure (T) is captured fully and does not squeeze out due to scissoring action of typical straight limb design ligation clips. Additionally, the non-interference of the distal portion of lower arm (112) with the proximal portion of distal hook (130) may reduce the applied moment, or torque, required to bring the upper surface of the distal portion of lower arm (112) into contact with the lower surface of upper arm (114).

After ligation clip (110) has assumed the partially closed state, jaws (J1, J2) may continue to apply a closure force to ligation clip (110) via protrusions (160, 162) to transition ligation clip (110) to the fully closed state, as shown in FIG. 1C. More particularly, the continued urging of protrusions (160, 162) toward each other by jaws (J1, J2) may cause arms (112, 114) to pivot toward each other about proximal hinge element (120) while allowing lower distal end (118) of lower arm (112) to be received by distal hook (130). In the example shown, distal end (118) of lower arm (112) is equipped with an angled or ramped surface (115) that, during closure to of ligation clip (110), facilitates the distal motion of distal end (118) of lower arm (112) into distal hook (130) through an interaction with the proximal face of trapezoidal protrusion (132) which both guides distal end (118) of lower arm (112) into distal hook (130) while also enabling sufficient deflection of distal hook (130), in a wedge like manner, until protrusions (132, 142) are fully seated into their respective recesses (134, 140). The distal motion and subsequent interlocking of the features may be achieved without significant force, or torque, being applied to clip (110) through the applicator end effector. Additionally, due to the wedge action of the lower arm distal end (118) on distal hook (130) of upper arm (114), no axial deflection of distal hook (130) may be necessary prior to engagement. In this regard, the upper surface of lower arm (112) may confront and/or be generally parallel to the lower surface of upper arm (114) to clamp tissue structure (T) therebetween. It will be appreciated that in some versions, at least a portion of the upper surface of lower arm (112) may engage at least a portion of the lower surface of upper arm (114), such as in the absence of tissue structure (T). The use of the distal hinge element (150) may facilitate the staged motion of clip (110) whereby distal end (118) of lower arm (112) pivots about an offset point of rotation relative to the apex of clip (110) located at proximal hinge element (120). Distal end (118) of lower arm (112) travels proximally prior to a reversal of motion into the distal direction that occurs once the upper surface of lower arm (112) contacts the lower surface of upper arm (114).After ligation clip (110) has assumed the fully closed state, jaws (J1, J2) may be released from ligation clip (110) and withdrawn while ligation clip (110) remains clamped over tissue structure (T). Once protrusions (132, 142) are seated into the respective recesses (134, 140), the reversal of axial motion of distal end (118) of lower arm (112) is prevented which prevents distal hinge element (150) from bending and releasing lower arm (112) from distal hook (130), thereby inhibiting ligation clip (110) from transitioning away from the fully closed state.

### B. Second Example of Surgical Ligation Clip

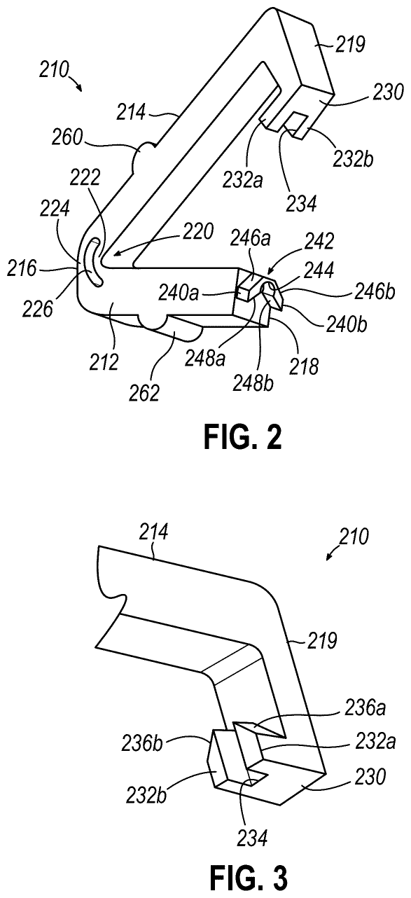

FIGS. 2-4B show another example of a surgical ligation clip (210). As shown, ligation clip (210) of this example includes a pair of arms (212, 214) including a lower arm (212) and an upper arm (214) that are joined together at a proximal end (216). In the example shown, lower arm (212) extends from proximal end (216) to a lower distal end (218), and upper arm (214) extends from proximal end (216) to an upper distal end (219). A deformable proximal hinge element (220) is defined at or near proximal end (216) by an inner hinge wall element (222) and an outer hinge wall element (224) that are spaced apart from each other by a generally C-shaped slot (226), such that arms (212, 214) may be movable relative to each other about proximal hinge element (220). In some versions, inner hinge wall element (222) and outer hinge wall element (224) may have different thicknesses from each other. Such different thicknesses may be selected to minimize the magnitude of closure force required for bending ligation clip (210) about proximal hinge element (220).

Upper arm (214) of the present example includes a distal hook (230) provided at or near upper distal end (219). Distal hook (230) is sized and configured to securely receive lower distal end (218) of lower arm (212) when ligation clip (210) is in a fully closed state. In this regard, distal hook (230) of the present example includes a pair of latching members in the form of first and second hook portions (232a, 232b) spaced apart from each other by a gap (234) and having corresponding first and second upper abutment surfaces (236a, 236b), which are each configured to interact with corresponding latching members of lower arm (212) to maintain ligation clip (210) in the fully closed state once ligation clip (210) reaches the fully closed state. To that end, lower arm (212) of the present example includes a pair of latching members in the form of first and second legs (240a, 240b) joined together at a deformable distal hinge element (242) that is defined by a generally C-shaped recess (244), and having corresponding first and second cam surfaces (246a, 246b) and first and second lower abutment surfaces (248a, 248b). First and second cam surfaces (246a, 246b) are configured to engage corresponding lower edges of first and second hook portions (232a, 232b), respectively, to pivot first and second legs (240a, 240b) inwardly toward each other about distal hinge element (242) for enabling first and second legs (240a, 240b) to pass through gap (234) during closure of ligation clip (210), while first and second lower abutment surfaces (248a, 248b) are configured to abut first and second upper abutment surfaces (236a, 236b), respectively, after first and second legs (240a, 240b) have passed through gap (234).

In the example shown, an upper protrusion (260) extends upwardly from an upper surface of upper arm (214) and a lower protrusion (262) extends downwardly from the lower surface of lower arm (212) slightly distal of proximal hinge element (220), which are each configured to be gripped by a corresponding jaw of a robotic applicator end effector (not shown). As described in greater detail below, the robotic applicator may transition ligation clip (210) between the open and fully closed states by applying one or more forces to the upper surface of upper protrusion (260) and the lower surface of lower protrusion (262).

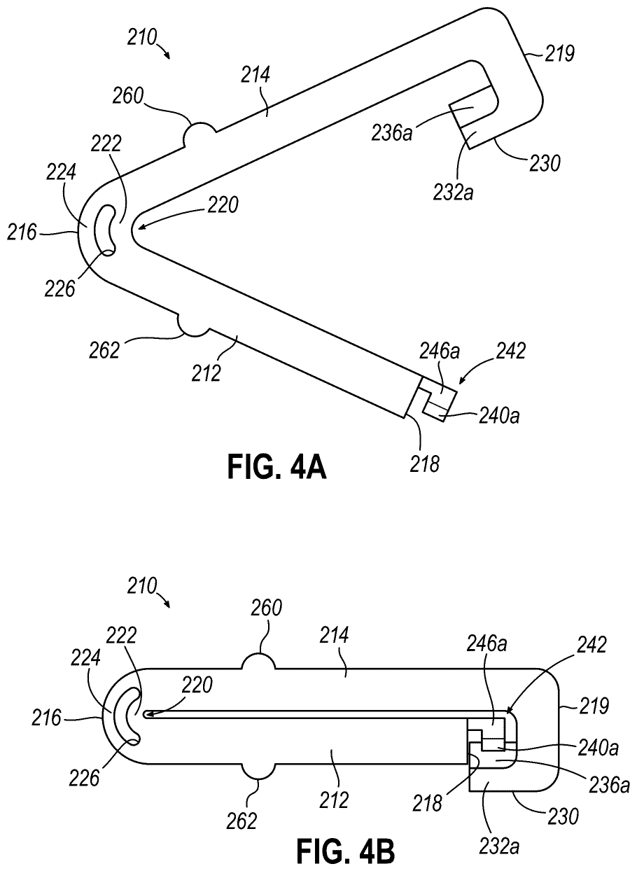

In this regard, ligation clip (110) may initially be in the open state, as shown in FIG. 4A. First and second jaws of the robotic applicator may then grip the corresponding surfaces of protrusions (260, 262) and maneuver ligation clip (210) to receive a tissue structure (e.g., vessel, etc.) (not shown) between arms (212, 214), and may further apply a closure force to ligation clip (210) via the corresponding surfaces of protrusions (260, 262) to transition ligation clip (210) to the fully closed state, as shown in FIG. 4B. More particularly, the urging of the upper surface of upper arm (214) and the lower surface of lower arm (212) toward each other by the jaws may cause arms (212, 214) to pivot toward each other about proximal hinge element (220) while allowing lower distal end (218) of lower arm (212) to be received by distal hook (230). In this regard, cam surfaces (246a, 246b) may engage the corresponding lower edges of hook portions (232a, 232b) to pivot first and second legs (240a, 240b) inwardly toward each other about distal hinge element (242), such that first and second legs (240a, 240b) may be passed through gap (234) of distal hook (230). The pivoting of cam surfaces (246a, 246b) may exert a lateral wedging force on the inner surfaces of hook portions (232a, 232b) and may result in the engagement of these features without axial deflection of hook portions (232a, 232b). The upper surface of lower arm (212) may then confront and/or be generally parallel to the lower surface of upper arm (214) to clamp the tissue structure therebetween. It will be appreciated that in some versions, at least a portion of the upper surface of lower arm (212) may engage at least a portion of the lower surface of upper arm (214).

After ligation clip (210) has assumed the fully closed state, the jaws may be released from ligation clip (210) and withdrawn while ligation clip (210) remains clamped over the tissue structure. As shown, when ligation clip (210) is in the fully closed state, lower abutment surfaces (248a, 248b) may be seated on the corresponding abutment surfaces (236a, 236b) to inhibit ligation clip (210) from transitioning away from the fully closed state.

### C. Third Example of Surgical Ligation Clip

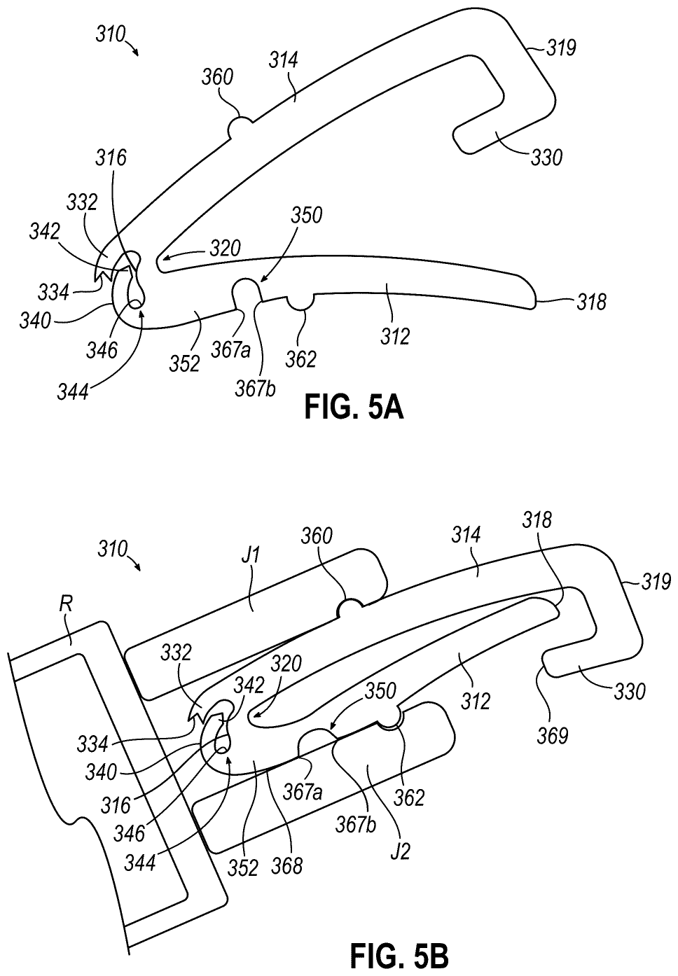

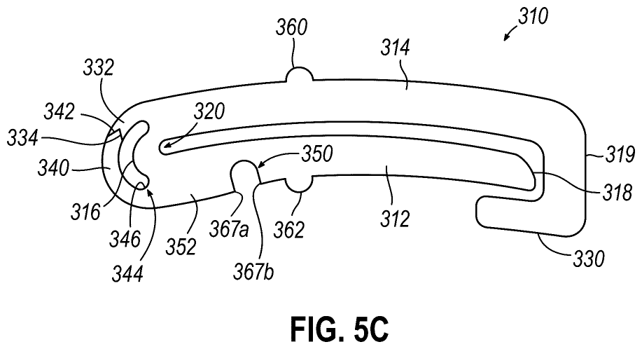

FIGS. 5A-5C show another example of a surgical ligation clip (310). As shown, ligation clip (310) of this example includes a pair of arms (312, 314) including a generally arcuate lower arm (312) and a generally arcuate upper arm (314) that are joined together at a proximal end (316). In the example shown, lower arm (312) extends from proximal end (316) to a lower distal end (318), and upper arm (314) extends from proximal end (316) to an upper distal end (319). A deformable proximal hinge element (320) is defined at or near proximal end (316), such that arms (312, 314) may be movable relative to each other about proximal hinge element (320).

Upper arm (314) of the present example includes a distal hook (330) provided at or near upper distal end (319). Distal hook (330) is sized and configured to securely receive lower distal end (318) of lower arm (312) when ligation clip (310) is in a fully closed state. In this regard, upper arm (314) of the present example includes an upper proximal finger (332) extending downwardly and proximally at or near proximal end (316) and having a latching member in the form of a generally triangular recess (334) extending upwardly from a lower surface of upper proximal finger (332), which is configured to interlock with corresponding latching members of lower arm (312) to maintain ligation clip (310) in the fully closed state once ligation clip (310) reaches the fully closed state. To that end, lower arm (312) of the present example includes a lower proximal finger (340) extending upwardly and distally at or near proximal end (316) and having a latching member in the form of a generally triangular protrusion (342) extending upwardly from an upper surface of lower proximal finger (340) and configured to be received by recess (334). In some versions, recess (334) and protrusion (342) may be sized and shaped relative to each other to provide a snap-fit therebetween. In the example shown, a deformable proximal-most hinge element (344) is defined proximal of proximal hinge element (320) by a generally C-shaped recess (346), such that lower proximal finger (340) may be movable relative to upper proximal finger (332) about proximal-most hinge element (344).

In the example shown, a deformable distal hinge element (350) is defined distal of proximal hinge element (320) by a generally C-shaped recess (352) extending upwardly from the lower surface of lower arm (312), sized such that an applied bending force that results in deflection of the distal portion of lower arm (312) that is distal to distal hinge element (350) is lower than what is required to cause bending of proximal hinge element (320). This difference in bending stiffness is such that the distal portion of lower arm (312) may be movable relative to a proximal portion of lower arm (312) about distal hinge element (350). Distal hinge element (350) may thereby allow ligation clip (310) to be movable to a partially closed state as described in greater detail below.

As shown, an upper protrusion (360) extends upwardly from an upper surface of upper arm (314) and a lower protrusion (362) extends downwardly from the lower surface of lower arm (312) slightly distal of distal hinge element (350), which are each configured to be gripped by a corresponding jaw of a robotic applicator (not shown) in a manner similar to that described above. As described in greater detail below, the robotic applicator may transition ligation clip (310) between the open, partially closed, and fully closed states by applying one or more forces to protrusions (360, 362). The use of distal hinge element (350) may facilitate the staged motion of clip (310) whereby distal end (318) of lower arm (312) pivots about an offset point of rotation relative to the apex of clip (310) located at proximal hinge element (320). Distal end (318) of lower arm (312) travels proximally prior to a reversal of motion into the distal direction that occurs once the upper surface of lower arm (312) contacts the lower surface of upper arm (314)

In this regard, ligation clip (310) may initially be in the open state, as shown in FIG. 5A. First and second jaws (J1, J2) of the robotic applicator may then grip the corresponding protrusions (360, 362) and maneuver ligation clip (310) to receive a tissue structure (e.g., vessel, etc.) (not shown) between arms (312, 314), and may further apply a closure force to ligation clip (310) via protrusions (360, 362) to transition ligation clip (310) to the partially closed state, as shown in FIG. 5B. More particularly, the urging of protrusions (360, 362) toward each other by the jaws (J1, J2) may cause the distal portion of lower arm (312) to pivot upwardly about distal hinge element (350) relative to the proximal portion of lower arm (312), such that lower distal end (318) of lower arm (312) may be brought proximal of distal hook (330) without colliding with distal hook (330). In the partially closed state, upper proximal finger (332) and lower proximal finger (340) may remain unaltered in relative position as the deflection of the distal portion of lower arm (312) about distal hinge element (350) may not cause deformation and/or deflection of proximal hinge element (320). In this position, corner features (367a, 367b) of distal hinge element (350) may contact an upper surface (368) of lower jaw (J2) of the applicator end effector.

After ligation clip (310) has assumed the partially closed state, the jaws may continue to apply a closure force to ligation clip (310) via protrusions (360, 362) to transition ligation clip (310) from the partially closed state shown in FIG. 5B to a fully closed state, as shown in FIG. 5C. More particularly, the continued closure of the upper and lower jaws (J1, J2) urges protrusions (360, 362) toward each other and applies pressure to corner features (367a, 367b) of distal hinge element (350) from the upper surface (368) of the lower jaw (J2). The localized pressure may cause proximal hinge element (320) to deform and/or deflect inwardly, while the distal portion of lower arm (312) that is distal to distal hinge element (350) may be returned to a generally axially aligned state with the original undeformed shape of lower arm (312). As proximal hinge element (320) is deformed into a bent condition, there may be a rotation of lower proximal finger (340) relative to upper proximal finger (332). More particularly, a proximal and downward rotation of lower proximal finger (340) may initially bring the proximal surface of lower proximal finger (340) into sliding contact with the inner surface of upper proximal finger (332). The sliding contact of lower proximal finger (340) with the inner surface of upper proximal finger (332) may continue throughout the full deflection of proximal hinge element (320) during closure until protrusion (342) of lower proximal finger (340) bypasses the lower distal edge of recess (334) of upper finger element (332). The continued closure of the jaws (J1, J2) may effectively cause arms (312, 314) to pivot toward each other about proximal hinge element (320) while allowing lower distal end (318) of lower arm (312) to be received by distal hook (330). In this regard, the upper surface of lower arm (312) may confront and/or be generally parallel to the lower surface of upper arm (314) to clamp the tissue structure therebetween. It will be appreciated that in some versions, at least a portion of the upper surface of lower arm (312) may engage at least a portion of the lower surface of upper arm (314). Additionally, the engagement of lower arm (312) with upper arm (314) is achieved without a distal axial deflection of distal end (319) or distal hook (330) as well as the resumption of the undeformed shape of distal hinge element (350) and a substantially undeformed lower arm (312).

After ligation clip (310) has assumed the fully closed state, the jaws may be released from ligation clip (310) and withdrawn while ligation clip (310) remains clamped over the tissue structure. As shown, when ligation clip (310) is in the fully closed state, recess (334) may receive protrusion (342) to inhibit ligation clip (310) from transitioning away from the fully closed state.

### D. Fourth Example of Surgical Ligation Clip

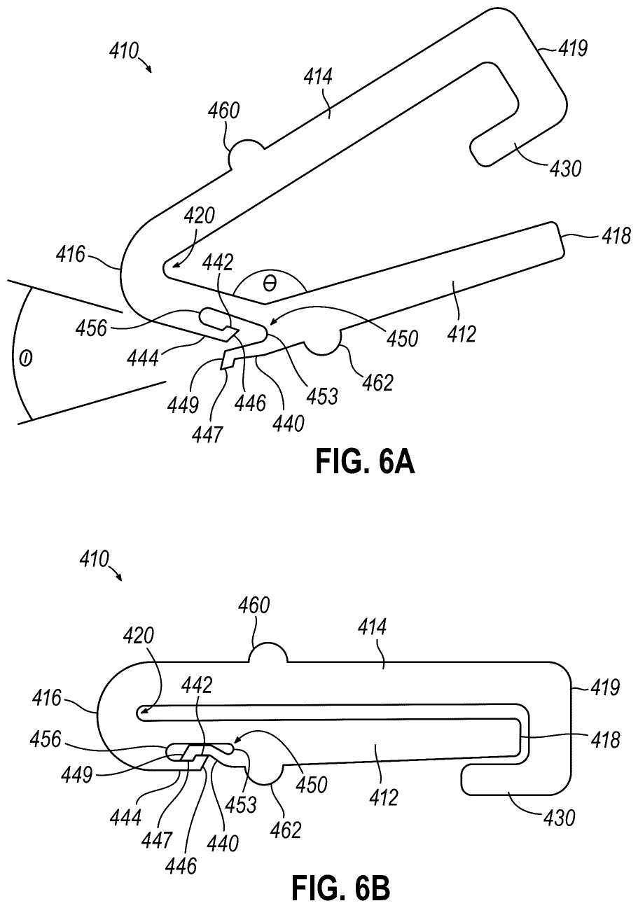

FIGS. 6A-6B show another example of a surgical ligation clip (410). As shown, ligation clip (410) of this example includes a pair of arms (412, 414) including a lower arm (412) and an upper arm (414) that are joined together at a proximal end (416). In the example shown, lower arm (412) extends from proximal end (416) to a lower distal end (418), and upper arm (414) extends from proximal end (416) to an upper distal end (419). A deformable proximal hinge element (420) is defined at or near proximal end (416), such that arms (412, 414) may be movable relative to each other about proximal hinge element (420).

Upper arm (414) of the present example includes a distal hook (430) provided at or near upper distal end (419). Distal hook (430) is sized and configured to securely receive lower distal end (418) of lower arm (412) when ligation clip (410) is in a fully closed and latched state. In this regard, lower arm (412) of the present example includes a pair of latching members in the form of a flexible tab (440) extending downwardly and/or proximally from a lower surface of lower arm (412) that is defined by a generally rectangular shaped arm with a downwardly-extending and/or proximally-extending trapezoidal shaped lower protrusion (447) having a cam surface (449); and a mating latching tab (444) disposed slightly proximally of flexible tab (440) that is defined by a generally rectangular shaped arm with an upwardly-extending and/or distally-extending trapezoidal shaped upper protrusion (442) having a cam surface (446), which are configured to interact with each other to maintain ligation clip (410) in the fully closed state once ligation clip (410) reaches the fully closed state. As shown, flexible tab (440) is configured with a hinge base (453), and mating latching tab (444) is similarly configured with a hinge base (456). Hinge bases (453, 456) are in opposition to each other and may collectively define a distal hinge element (450). Cam surfaces (446, 449) are configured to facilitate a bypassing engagement of flexible tabs (440, 444) to deflect flexible tab (440) past the opposing mating latching tab (444) during closure of ligation clip (410), and upper protrusion (442) is configured to then be located below lower protrusion (447) to securely capture the bent flexible tab (440).

In the embodiment shown, lower arm (412) has a proximal portion that extends from proximal end (416) of ligation clip (410) to distal hinge element (450), and a distal portion that extends from distal hinge element (450) to distal end (418) of lower arm (412) at a first predetermined angle (θ), at least when in an unstressed state. In the unstressed state, flexible tab (440) extends downwardly and proximally at a second predetermined angle (Φ) relative to mating latching tab (444),

As shown, an upper protrusion (460) extends upwardly from an upper surface of upper arm (414) and a lower protrusion (462) extends downwardly from the lower surface of lower arm (412) slightly distal of distal hinge element (450), which are each configured to be gripped by a corresponding jaw of a robotic applicator (not shown) in a manner similar to that described above. As described in greater detail below, the robotic applicator may transition ligation clip (410) between the open, partially closed, and fully closed states by applying one or more forces to protrusions (460, 462).

In this regard, ligation clip (410) may initially be in the open state, as shown in FIG. 6A. First and second jaws of the robotic applicator may then grip the corresponding protrusions (460, 462) and maneuver ligation clip (410) to receive a tissue structure (e.g., vessel, etc.) (not shown) between arms (412, 414), and may further apply a closure force to ligation clip (410) via protrusions (460, 462) to transition ligation clip (410) to the partially closed state (not shown More particularly, the urging of protrusions (460, 462) toward each other by the jaws may cause the distal portion of lower arm (412) to pivot upwardly about distal hinge element (450) relative to the proximal portion of lower arm (412), such that lower distal end (418) of lower arm (412) may be brought proximal of distal hook (430) without colliding with distal hook (430), such as in a manner similar to that shown and described above in connection with FIG. 1B. In some versions, flexible tab (440) may bend upwardly relative to mating latch (444) when ligation clip (410) is transitioned from the partially closed state to the fully compressed or closed state.

After ligation clip (410) has assumed the partially closed state, the jaws may continue to apply a closure force to ligation clip (410) via protrusions (460, 462) to transition ligation clip (410) to the fully closed state, as shown in FIG. 6B. More particularly, the continued urging of protrusions (460, 462) toward each other by the jaws may cause arms (412, 414) to pivot toward each other about proximal hinge element (420) while allowing lower distal end (418) of lower arm (412) to be received by distal hook (430). As force is applied to protrusions (460,462), the distal portion of lower arm (412) that extends distally from distal hinge element (450) to distal end (418) pivots radially about distal hinge element (450), and distal end (418) of lower arm (412) moves proximally relative to proximal hinge element (420) until distal end (418) contacts the lower surface of upper arm (414), and continued application of closure force to protrusions (460,462) forces distal end (418) distally along the lower surface of upper arm (414) until lower arm (412) is deformed into a generally straight condition and distal hinge element (450) is in a deformed state.

After ligation clip (410) has assumed the fully closed state, the jaws may be released from ligation clip (410) and withdrawn while ligation clip (410) remains clamped over the tissue structure. As shown, when ligation clip (410) is in the fully closed state, mating latch (444) may be in a deflected position mating with flexible tab (440) while protrusions (446, 447) may be engaged with each other in a mated and locked position to inhibit ligation clip (410) from transitioning away from the fully closed state.

### E. Fifth Example of Surgical Ligation Clip

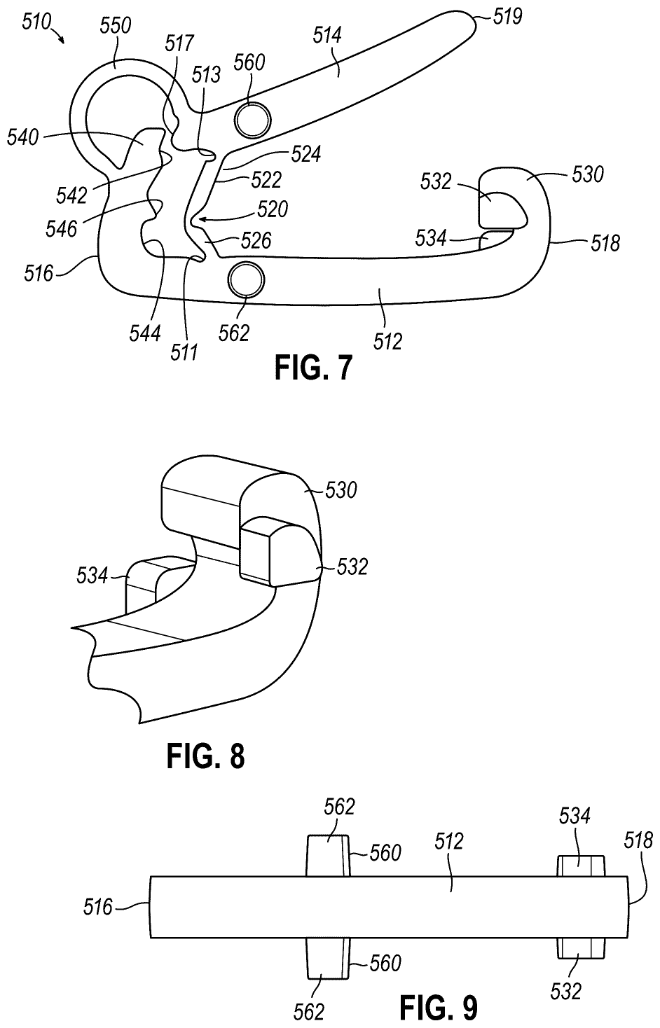

FIGS. 7-10D show another example of a surgical ligation clip (510). As shown, ligation clip (510) of this example includes a pair of arms (512, 514) including a lower arm (512) and an upper arm (514). In the example shown, lower arm (512) extends from a lower proximal end (516) to a lower distal end (518), and upper arm (514) extends from an upper proximal end (517) to an upper distal end (519). A deformable intermediate hinge element (520) is defined at or near a midpoint of a thin column (522) that joins arms (512, 514) together at or near the respective proximal ends (516, 517) and that has an upper column portion (524) and a lower column portion (526), such that arms (512, 514) may be movable relative to each other about intermediate hinge element (520). The upper end of upper column portion (524) is connected to the lower side of upper arm (514) through a reduced thickness upper hinge element (513) and the lower end of lower column portion (526) is connected to the upper side of lower arm (512) through a lower hinge element (511), and the thickness of lower hinge element (511) is greater than the thickness(es) of upper hinge element (513) and/or intermediate hinge element (520). In the example shown, column portions (524, 526) are connected with an obtuse arcuate bend to define intermediate hinge element (520). In some versions, lower column portion (526) may have a length less than that of upper column portion (524).

Lower arm (512) of the present example includes a distal hook (530) provided at or near lower distal end (518). Distal hook (530) is sized and configured to securely receive upper distal end (519) of upper arm (514) when ligation clip (510) is in a fully closed state. In this regard, lower arm (512) of the present example includes a pair of engagement members in the form of upper and lower laterally-outwardly extending sidewalls (532, 534), which are configured to cooperate with each other to maintain ligation clip (510) in a generally aligned and laterally restricted state once ligation clip (510) reaches the fully closed state. More particularly, sidewalls (532, 534) are configured to laterally flank upper distal end (519) of upper arm (514). While sidewalls (532,534) are positioned on each side of distal hook (530), they are positioned to only cover a portion of the open side of distal hook (530), the coverage being half or less than half of the open width of distal hook (530). Sidewalls (532,534) are also positioned such that when viewed though the side of distal hook (530) they do not overlap, such that sidewalls (532, 534) may be produced through simple straight pull mold design without complex slide features, thereby enabling less costly manufacturing tooling. Lower arm (512) of the present example further includes a proximal upwardly-extending cam leg (540) provided at or near lower proximal end (516). Cam leg (540) includes an upper indent (542) extending proximally from a distal surface of cam leg (540) and a receptacle (544) that is defined by a lower detent (546) extending distally from the distal surface of cam leg (540), which are configured to interact with upper proximal end (517) to assist with transitioning ligation clip (510) toward the fully closed state and/or maintain ligation clip (510) in the fully closed state once ligation clip (510) reaches the fully closed state.

In the example shown, arms (512, 514) are further joined together by a generally arcuate proximal hinge element (550) extending from a proximal surface of cam leg (540) at or near a midpoint thereof to an upper surface of upper arm (514) at or near upper proximal end (517), and spanning through an included angle of approximately 210°.

As shown, a pair of upper protrusions (560) extend laterally outwardly from respective side surfaces of upper arm (514) and a pair of lower protrusions (562) extend laterally outwardly from respective side surfaces of lower arm (512) slightly distal of proximal hinge element (520), which are each configured to be gripped by a corresponding jaw of a robotic applicator (not shown) in a manner similar to that described above. As described in greater detail below, the robotic applicator may transition ligation clip (510) between the open, partially closed, and fully closed states by applying one or more forces to protrusions (560, 562).

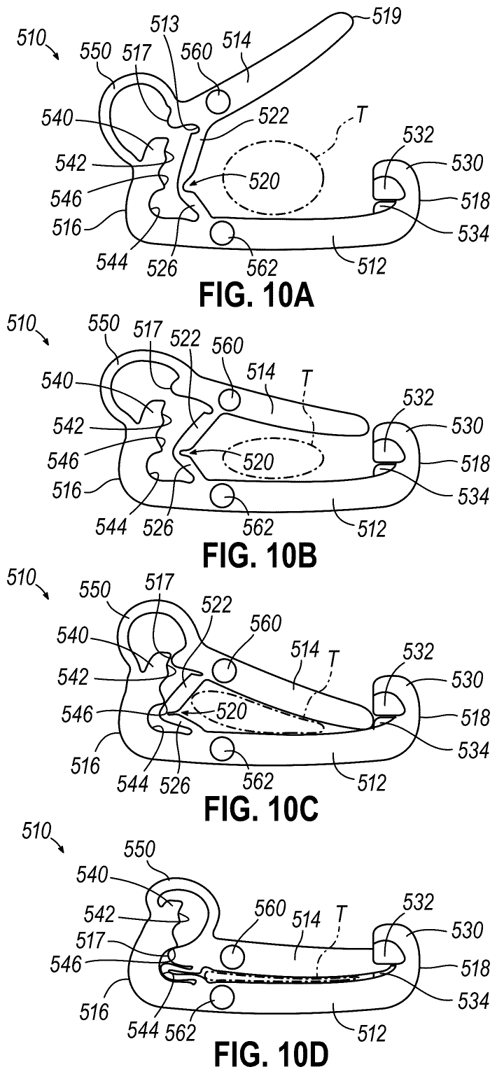

In this regard, ligation clip (510) may initially be in the open state, as shown in FIG. 10A. First and second jaws of the robotic applicator may then grip the corresponding protrusions (560, 562) and maneuver ligation clip (510) to receive a tissue structure (e.g., vessel, etc.) (T) between arms (512, 514), and may further apply a closure force to ligation clip (510) via protrusions (560, 562) to transition ligation clip (510) to the first partially closed state, as shown in FIG. 10B. More particularly, the urging of protrusions (560, 562) toward each other by the jaws may cause upper distal end (519) of upper arm (514) to pivot relative to upper and lower column portions (524, 526) about upper hinge element (513) and move downwardly toward distal hook (530). The rotation of upper arm (514) may be induced through the applied force to protrusions (560,562) and the retention of proximal end (517) of upper arm (514) with the upper portion of arcuate proximal hinge element (550). In some versions, column portions (524, 526) may bend proximally at upper hinge element (513) as well as a secondary bending of intermediate hinge element (520) when ligation clip (510) is transitioned to the first partially closed state. The effect of the loading of protrusions (560,562) is counteracted by the upper portion of arcuate proximal hinge element (550) to control the pivot of upper arm (514) about upper hinge element (513), in a manner similar to a beam in three-point loading. The pivoting results in an axial proximal displacement of distal end (519) of upper arm (514) relative to lower proximal end (516) of clip (510) until the lower surface of distal end (519) of upper arm (514) makes contact with the upper surface of lower arm (512). Upon increased loading of protrusions (560,562), upper proximal end (517) of upper arm (514) may be received by upper indent (542) of cam leg (540) when ligation clip (510) is transitioned to the first partially closed state.

After ligation clip (510) has assumed the first partially closed state, the jaws may continue to apply a closure force to ligation clip (510) via protrusions (560, 562) to transition ligation clip (510) to the second partially closed state, as shown in FIG. 10C. More particularly, the continued urging of protrusions (560, 562) toward each other by the jaws may cause upper distal end (519) of upper arm (514) to move further downwardly and/or slightly distally, such that upper distal end (519) may be brought proximal of distal hook (530) and/or captured by distal hook (530). In some versions, column portions (524, 526) may bend proximally at intermediate hinge element (520) when ligation clip (510) is transitioned to the second partially closed state. In addition, or alternatively, upper proximal end (517) of upper arm (514) may pivot within and slide downwardly against the distal surface of upper indent (542) of leg (540) when ligation clip (510) is transitioned to the second partially closed state.

After ligation clip (510) has assumed the second partially closed state, the jaws may continue to apply a closure force to ligation clip (510) via protrusions (560, 562) to transition ligation clip (510) to the fully closed state, as shown in FIG. 10D. More particularly, the continued urging of protrusions (560, 562) toward each other by the jaws may cause upper proximal end (517) of upper arm (514) and/or proximal hinge element (520) to move by sliding downwardly into receptacle (544) while allowing upper distal end (519) of upper arm (514) to move distally, relative to lower proximal end (516), to be received by distal hook (530). In this regard, the upper surface of lower arm (512) may contact and/or be generally parallel to the lower surface of upper arm (514) to clamp tissue structure (T) therebetween. Additionally, the pressure of the clamped tissue structure (T) within clip (510) in the fully closed state may force the upper surface of upper arm (514) against the lower inner surface of the upper portion of distal hook (530). It may therefore be possible to control the maximum gap of the fully closed clip (510) through the sizing of the opening of distal hook (530) and thereby produce the desired tissue clamping effect. It will be appreciated that in some versions, at least a portion of the upper surface of lower arm (512) may engage at least a portion of the lower surface of upper arm (514), such as in the absence of tissue structure (T). In some versions, column portions (524, 526) may bend proximally at intermediate hinge element (520) when ligation clip (510) is transitioned to the fully closed state.

After ligation clip (510) has assumed the fully closed state, the jaws may be released from ligation clip (510) and withdrawn while ligation clip (510) remains clamped over the tissue structure. As shown, when ligation clip (510) is in the fully closed state, receptacle (544) may receive upper proximal end (517) of upper arm (514) and/or proximal hinge element (520) to inhibit ligation clip (510) from transitioning away from the fully closed state.

### F. Sixth Example of Surgical Ligation Clip

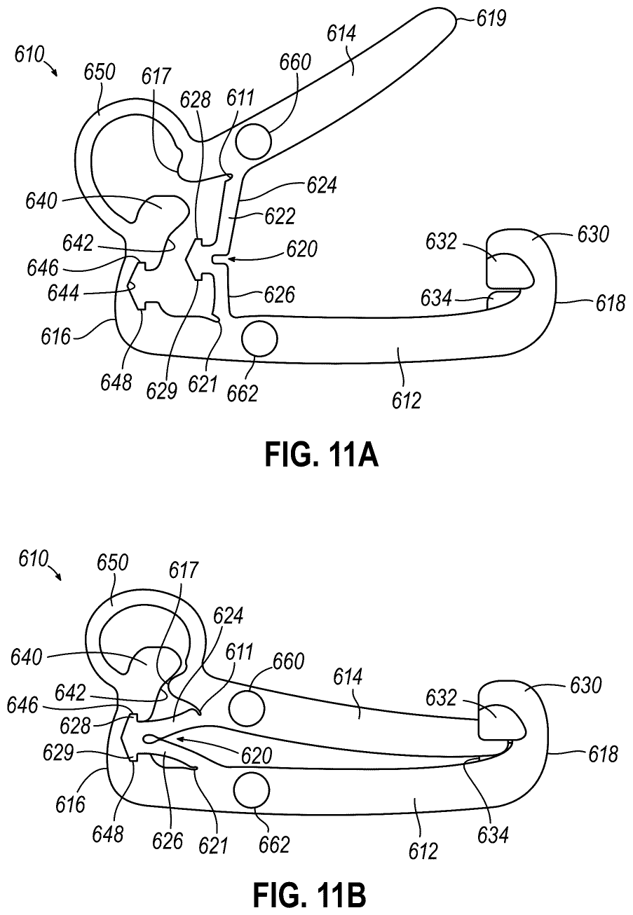

FIGS. 11A-11B show another example of a surgical ligation clip (610). As shown, ligation clip (610) of this example includes a pair of arms (612, 614) including a lower arm (612) and an upper arm (614). In the example shown, lower arm (612) extends from a lower proximal end (616) to a lower distal end (618), and upper arm (614) extends from an upper proximal end (617) to an upper distal end (619). A deformable intermediate hinge element (620) is defined at or near an intersection of an upper column portion (624) and a lower column portion (626) which in cooperation form a unitary column (622) that joins arms (612, 614) together at or near the respective proximal ends (616, 617). Upper column portion (624) is connected to the lower side of upper arm (614) through a thin walled upper hinge element (611), and lower column portion (626) is connected to the upper surface of lower arm (612) through a thin walled hinge element (621). Arms (612, 614) may be movable relative to each other about arcuate proximal hinge element (650). Upper and lower column portions (624, 626) of the present example include a pair of latching members in the form of upper and lower flexible tabs (628, 629), the purposes of which are described below.

Lower arm (612) of the present example includes a distal hook (630) provided at or near lower distal end (618). Distal hook (630) is sized and configured to securely receive upper distal end (619) of upper arm (614) when ligation clip (610) is in a fully closed state. In this regard, lower arm (612) of the present example includes a pair of engagement members in the form of upper and lower laterally-outwardly extending sidewalls (632, 634), which are configured to cooperate with each other to maintain ligation clip (610) and, more particularly, upper and lower arms (614,612), in the fully closed and generally laterally aligned state once ligation clip (610) reaches the fully closed state. More particularly, sidewalls (632, 634) are configured to laterally flank upper distal end (619) of upper arm (614). Lower arm (612) of the present example further includes a proximal upwardly-extending cam leg (640) provided at or near lower proximal end (616). Cam leg (640) includes an upper indent (642) extending proximally from a distal surface of cam leg (640) and a receptacle (644) extending proximally from the distal surface of cam leg (640) and having upper and lower recesses (646, 648), which are configured to interact with upper proximal end (617) and/or flexible tabs (628, 629) to assist with transitioning ligation clip (610) toward the fully closed state and/or maintain ligation clip (610) in the fully closed state once ligation clip (610) reaches the fully closed state.

In the example shown, arms (612, 614) are further joined together by a generally arcuate proximal hinge element (650) extending from a proximal surface of leg (640) at or near a midpoint thereof to an upper surface of upper arm (614) at or near upper proximal end (617), and spanning through an included angle of approximately 210°.

As shown, a pair of upper protrusions (660) extend laterally outwardly from respective side surfaces of upper arm (614) and a pair of lower protrusions (662) extend laterally outwardly from respective side surfaces of lower arm (612) slightly distal of proximal hinge element (620), which are each configured to be gripped by a corresponding jaw of a robotic applicator (not shown) in a manner similar to that described above. As described in greater detail below, the robotic applicator may transition ligation clip (610) between the open, partially closed, and fully closed states by applying one or more forces to protrusions (660, 662).

In this regard, ligation clip (610) may initially be in the open state, as shown in FIG. 11A. First and second jaws of the robotic applicator may then grip the corresponding protrusions (660, 662) and maneuver ligation clip (610) to receive a tissue structure (e.g., vessel, etc.) (not shown) between arms (612, 614), and may further apply a closure force to ligation clip (610) via protrusions (660, 662) to transition ligation clip (610) to the first partially closed state (not shown). More particularly, the urging of protrusions (660, 662) toward each other by the jaws may cause upper distal end (619) of upper arm (614) to pivot downwardly toward the upper surface of lower arm (612) and in a generally axial proximal direction, away from the proximal surface of distal hook (630), such as in a manner similar to that shown and described above in connection with FIG. 10B. In some versions, upper and lower column portions (624, 626) may bend proximally at intermediate hinge element (620) when ligation clip (610) is transitioned to the first partially closed state. Since upper column portion (624) is longer than lower column portion (626), once upper hinge element (611), intermediate hinge element (620), and lower hinge element (621) have been deflected, upper arm (614) is advanced distally relative to lower arm (612) in the final captured position. In addition, or alternatively, upper proximal end (617) of upper arm (614) may be received by upper indent (642) of leg (640) when ligation clip (610) is transitioned to the first partially closed state.

After ligation clip (610) has assumed the first partially closed state, the jaws may continue to apply a closure force to ligation clip (610) via protrusions (660, 662) to transition ligation clip (610) to the second partially closed state (not shown). More particularly, the continued urging of protrusions (660, 662) toward each other by the jaws may cause upper distal end (619) of upper arm (614) to move further downwardly and/or slightly distally, such that upper distal end (619) may be brought proximal of distal hook (630) and/or captured by distal hook (630), such as in a manner similar to that shown and described above in connection with FIG. 10C. In some versions, column (622) may bend proximally at proximal hinge element (620) when ligation clip (610) is transitioned to the second partially closed state. In addition, or alternatively, upper proximal end (617) of upper arm (614) may pivot within upper indent (642) of leg (640) when ligation clip (610) is transitioned to the second partially closed state.

After ligation clip (610) has assumed the second partially closed state, the jaws may continue to apply a closure force to ligation clip (610) via protrusions (660, 662) to transition ligation clip (610) to the fully closed state, as shown in FIG. 11B. More particularly, the continued urging of protrusions (660, 662) toward each other by the jaws may cause upper proximal end (617) of upper arm (614) and/or proximal hinge element (620) to move by sliding downwardly into receptacle (644) while allowing upper distal end (619) of upper arm (614) to move distally, relative to lower proximal end (616) to be received by distal hook (630). In this regard, the upper surface of lower arm (612) may confront and/or be generally parallel to the lower surface of upper arm (614) to clamp tissue structure (T) therebetween. It will be appreciated that in some versions, at least a portion of the upper surface of lower arm (612) may engage at least a portion of the lower surface of upper arm (614), such as in the absence of tissue structure (T). In some versions, column (622) may bend proximally at intermediate hinge element (620) when ligation clip (610) is transitioned to the fully closed state.

After ligation clip (610) has assumed the fully closed state, the jaws may be released from ligation clip (610) and withdrawn while ligation clip (610) remains clamped over the tissue structure. As shown, when ligation clip (610) is in the fully closed state, receptacle (644) may receive proximal hinge element (620), with recesses (646, 648) receiving the corresponding flexible tabs (628, 629) to inhibit ligation clip (610) from transitioning away from the fully closed state.

### G. Seventh Example of Surgical Ligation Clip

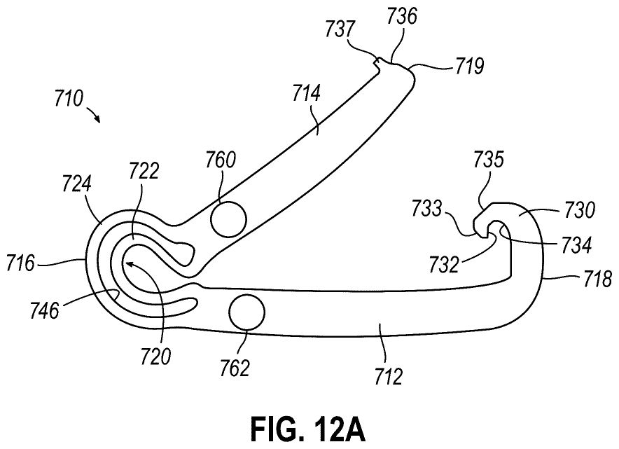

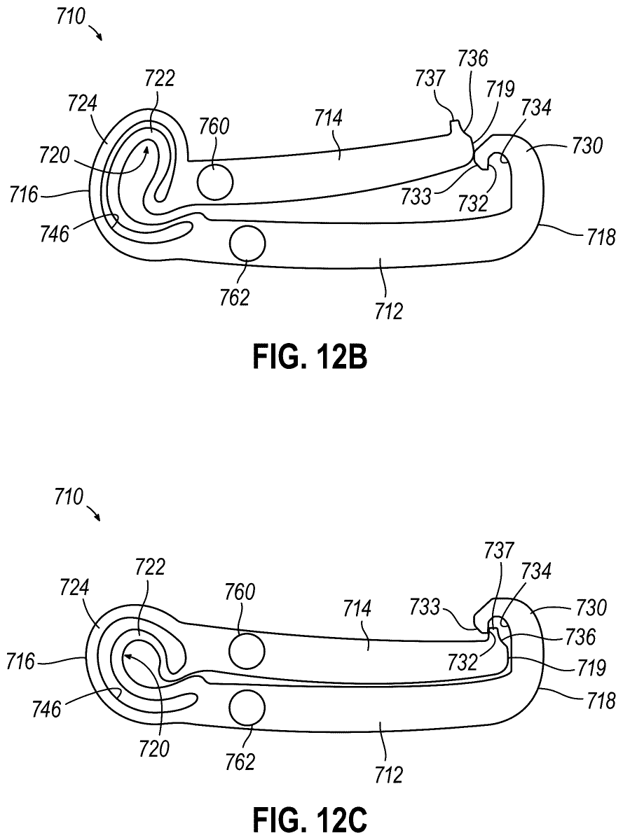

FIGS. 12A-12C show another example of a surgical ligation clip (710). As shown, ligation clip (710) of this example includes a pair of arms (712, 714) including a lower arm (712) and an upper arm (714) that are joined together at a proximal end (716). In the example shown, lower arm (712) extends from proximal end (716) to a lower distal end (718), and upper arm (714) extends from proximal end (716) to an upper distal end (719). A deformable proximal hinge element (720) is defined at or near proximal end (716) by an inner thin webbing (722) and an outer thin webbing (724) that are parallel to each other and spaced apart from each other by a generally C-shaped slot (726), such that arms (712, 714) may be both pivotable and translatable relative to each other via proximal hinge element (720).

Lower arm (712) of the present example includes a distal hook (730) provided at or near lower distal end (718). Distal hook (730) is sized and configured to securely receive upper distal end (719) of upper arm (714) when ligation clip (710) is in a fully closed state. As shown, distal hook (730) is generally “J” shaped with a trapezoidal protrusion that extends downwardly from the inner surface of distal hook (730) and a lower angular face (733) that is located on the lower proximal side of distal hook (730), and also has an upper angular face (735) that is located on the upper proximal side of distal hook (730). Distal end (719) of upper arm (714) has a distal angular face (736) and a trapezoidal shaped protrusion extending upwardly away from the upper surface of upper arm (714).

As shown, a pair of upper protrusions (760) extend laterally outwardly from respective side surfaces of upper arm (714) and a pair of lower protrusions (762) extend laterally outwardly from respective side surfaces of lower arm (712) slightly distal of proximal hinge element (720), which are each configured to be gripped by a corresponding jaw of a robotic applicator (not shown) in a manner similar to that described above. As described in greater detail below, the robotic applicator may transition ligation clip (710) between the open, partially closed, and fully closed states by applying one or more forces to protrusions (760, 762). The upper jaw of the robotic applicator end effector (not shown) may be equipped with a long pocket to engage with the upper protrusion (760) which accommodates any axial translation of upper protrusion (760) during the course of clip application.

In this regard, ligation clip (710) may initially be in the open state, as shown in FIG. 12A. First and second jaws of the robotic applicator may then grip the corresponding protrusions (760, 762) and maneuver ligation clip (710) to receive a tissue structure (e.g., vessel, etc.) (not shown) between arms (712, 714), and may further apply a closure force to ligation clip (710) via protrusions (760, 762) to transition ligation clip (710) to the partially closed state, as shown in FIG. 12B. More particularly, the urging of protrusions (760, 762) toward each other by the jaws may cause arms (712, 714) to pivot toward each other about proximal hinge element (720), such that upper distal end (719) of upper arm (714) may cammingly engage upper proximal face (735) of distal hook (730) to translate upper arm (714) proximally relative to lower arm (714) along a longitudinal axis that is generally parallel to lower arm (714).

After ligation clip (710) has assumed the partially closed state, the jaws may continue to apply a closure force to ligation clip (710) via protrusions (760, 762) to transition ligation clip (710) to the fully closed state, as shown in FIG. 12C. More particularly, the continued urging of protrusions (760, 762) toward each other by the jaws may cause arms (712, 714) to continue to pivot toward each other about proximal hinge element (720) while allowing upper distal end (719) of upper arm (714) to cammingly engage proximal angular face (735) of distal hook (730) so that upper arm (714) may translate proximally relative to lower arm (714) along the longitudinal axis and cause upper distal end (719) to travel past distal hook (730) without significantly deflecting distal hook (730). Continued closure of the protrusions (760, 762), coupled with the long pocket on the robotic end effector causes distal end (719) of upper arm (714) to fully pass by lower trapezoidal protrusion (733) of distal hook (730), at which point upper arm (714) translates distally in slidable engagement with lower arm (712) until upper trapezoidal protrusion (737) is in mated engagement with lower protrusion (733). The mated engagement of upper protrusion (737) within a distal receiver pocket (734) of distal hook (730) serves to lock upper arm (714) into engagement with lower arm (712). In this regard, the upper surface of lower arm (712) may confront and/or be generally parallel to the lower surface of upper arm (714) to clamp the tissue structure therebetween. It will be appreciated that in some versions, at least a portion of the upper surface of lower arm (712) may engage at least a portion of the lower surface of upper arm (714).

After ligation clip (710) has assumed the fully closed state, the jaws may be released from ligation clip (710) and withdrawn while ligation clip (710) remains clamped over the tissue structure.

### II. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A surgical ligation clip comprising: (a) a first arm extending from a proximal end to a first distal end, wherein the first arm includes a distal hook at the first distal end; and (b) a second arm extending from the proximal end to a second distal end, wherein the second arm is pivotable relative to the first arm about a proximal hinge to clamp tissue between the first and second arms, wherein the second arm includes: (i) a proximal portion, and (ii) a distal portion, wherein the distal portion is pivotable relative to the proximal portion about a distal hinge.

### Example 2

The surgical ligation clip of Example 1, wherein the second arm is configured to be pivoted away from the first arm about the proximal hinge to define an open state of the surgical ligation clip, wherein the distal portion is configured to be pivoted toward the first arm about the distal hinge to define a partially closed state of the surgical ligation, and wherein the second arm is configured to be pivoted toward the first arm about the proximal hinge to define a fully closed state of the surgical ligation clip.

### Example 3

The surgical ligation clip of Example 2, wherein the first and second arms are configured to be spaced apart from each other to receive the tissue between the first and second arms when the surgical ligation clip is in the open state.

### Example 4

The surgical ligation clip of any of Examples 2 through 3, wherein the second distal end is configured to be positioned proximal of the distal hook when the surgical ligation clip is in the partially closed state.

### Example 5

The surgical ligation clip of any of Examples 2 through 4, wherein the second distal end is configured to remain disengaged from the distal hook when the surgical ligation clip is transitioned from the open state to the partially closed state.

### Example 6

The surgical ligation clip of any of Examples 2 through 5, wherein the second distal end is configured to be received by the distal hook when the surgical ligation clip is in the fully closed state.

### Example 7

The surgical ligation clip of any of Examples 2 through 6, further comprising at least one engagement member configured to inhibit the surgical ligation clip from transitioning away from the fully closed state.

### Example 8

The surgical ligation clip of Example 7, wherein the at least one engagement member includes a recess provided on one of the first or second arms and a protrusion provided on the other of the first or second arms, wherein the recess and the protrusion are configured to interlock with each other to inhibit the surgical ligation clip from transitioning away from the fully closed state.

### Example 9

The surgical ligation clip of any of Examples 7 through 8, wherein the at least one engagement member is distal of each of the proximal and distal hinges.

### Example 10

The surgical ligation clip of any of Examples 7 through 8, wherein the at least one engagement member is proximal of each of the proximal and distal hinges.

### Example 11

The surgical ligation clip of any of Examples 7 through 8, wherein the at least one engagement member is proximal of the distal hinge and distal of the proximal hinge.

### Example 12

The surgical ligation clip of any of Examples 1 through 11, wherein the proximal hinge is defined by first and second walls spaced apart from each other by a slot.

### Example 13

The surgical ligation clip of Example 12, wherein the first and second walls have different thicknesses from each other.

### Example 14

The surgical ligation clip of any of Examples 1 through 13, wherein the first and second arms include first and second protrusions, respectively, configured to be gripped by corresponding jaws of a robotic applicator.

### Example 15

The surgical ligation clip of any of Examples 1 through 14, wherein each of the first and second arms includes a bioabsorbable material.

### Example 16

A surgical ligation clip comprising: (a) a first arm extending from a proximal end to a first distal end, wherein the first arm includes a distal hook at the first distal end, wherein the distal hook includes first and second distal hook portions spaced apart from each other by a gap; and (b) a second arm extending from the proximal end to a second distal end, wherein the second arm is pivotable relative to the first arm about a proximal hinge to clamp tissue between the first and second arms, wherein the second arm includes first and second distal legs pivotable relative to each other about a distal hinge.

### Example 17

The surgical ligation clip of Example 16, wherein the second arm is configured to be pivoted away from the first arm about the proximal hinge to define an open state of the surgical ligation clip, and wherein the second arm is configured to be pivoted toward the first arm about the proximal hinge to define a fully closed state of the surgical ligation clip.

### Example 18

The surgical ligation clip of Example 17, wherein the first and second distal hook portions are configured to cammingly engage the first and second distal legs, respectively, to pivot the first and second distal legs toward each other about the distal hinge for allowing the first and second distal legs to pass through the gap when the surgical ligation clip is transitioned from the open state to the fully closed state, wherein the first and second distal hook portions are configured to abut the first and second distal legs, respectively, to inhibit the surgical ligation clip from transitioning away from the fully closed state.

### Example 19

A surgical ligation clip comprising: (a) a first arm extending from a first proximal end to a first distal end, wherein the first arm includes a distal hook at the first distal end; (b) a second arm extending from a second proximal end to a second distal end, wherein the second arm includes a distal hook at the second distal end, wherein the second arm is movable relative to the first arm to clamp tissue between the first and second arms; (c) a column extending between the first and second arms, wherein the column includes first and second column portions pivotable relative to each other about a hinge; and (d) an arcuate hinge extending between the first and second arms proximally of the columns.

### Example 20

The surgical ligation clip of Example 19, wherein the distal hook includes a pair of sidewalls configured to laterally flank the second distal end of the second arm when the surgical ligation clip is in a fully closed state to inhibit lateral misalignment of the first and second arms.

### III. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, California. Similarly, those of ordinary skill in the art will recognize that various teachings herein may be readily combined with various teachings of any of the following: U.S. Pat. No. 5,792,135, entitled “Articulated Surgical Instrument For Performing Minimally Invasive Surgery With Enhanced Dexterity and Sensitivity,” issued Aug. 11, 1998, the disclosure of which is incorporated by reference herein; U.S. Pat. No. 8,783,541, entitled “Robotically-Controlled Surgical End Effector System,” issued Jul. 22, 2014, the disclosure of which is incorporated by reference herein; U.S. Pat. No. 8,479,969, entitled “Drive Interface for Operably Coupling a Manipulatable Surgical Tool to a Robot,” issued Jul. 9, 2013; U.S. Pat. No. 8,800,838, entitled “Robotically-Controlled Cable-Based Surgical End Effectors,” issued Aug. 12, 2014, the disclosure of which is incorporated by reference herein; and/or U.S. Pat. No. 8,573,465, entitled “Robotically-Controlled Surgical End Effector System with Rotary Actuated Closure Systems,” issued Nov. 5, 2013, the disclosure of which is incorporated by reference herein.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A surgical ligation clip comprising:
(a) a first arm extending from a proximal end to a first distal end, wherein the first arm includes a distal hook at the first distal end; and
(b) a second arm extending from the proximal end to a second distal end, wherein the second arm is pivotable relative to the first arm about a proximal hinge to clamp tissue between the first and second arms, wherein the second arm includes:
(i) a proximal portion, and
(ii) a distal portion, wherein the distal portion is pivotable relative to the proximal portion about a distal hinge.

2. The surgical ligation clip of claim 1, wherein the second arm is configured to be pivoted away from the first arm about the proximal hinge to define an open state of the surgical ligation clip, wherein the distal portion is configured to be pivoted toward the first arm about the distal hinge to define a partially closed state of the surgical ligation, and wherein the second arm is configured to be pivoted toward the first arm about the proximal hinge to define a fully closed state of the surgical ligation clip.

3. The surgical ligation clip of claim 2, wherein the first and second arms are configured to be spaced apart from each other to receive the tissue between the first and second arms when the surgical ligation clip is in the open state.

4. The surgical ligation clip of claim 2, wherein the second distal end is configured to be positioned proximal of the distal hook when the surgical ligation clip is in the partially closed state.

5. The surgical ligation clip of claim 2, wherein the second distal end is configured to remain disengaged from the distal hook when the surgical ligation clip is transitioned from the open state to the partially closed state.

6. The surgical ligation clip of claim 2, wherein the second distal end is configured to be received by the distal hook when the surgical ligation clip is in the fully closed state.

7. The surgical ligation clip of claim 2, further comprising at least one engagement member configured to inhibit the surgical ligation clip from transitioning away from the fully closed state.

8. The surgical ligation clip of claim 7, wherein the at least one engagement member includes a recess provided on one of the first or second arms and a protrusion provided on the other of the first or second arms, wherein the recess and the protrusion are configured to interlock with each other to inhibit the surgical ligation clip from transitioning away from the fully closed state.

9. The surgical ligation clip of claim 7, wherein the at least one engagement member is distal of each of the proximal and distal hinges.

10. The surgical ligation clip of claim 7, wherein the at least one engagement member is proximal of each of the proximal and distal hinges.

11. The surgical ligation clip of claim 7, wherein the at least one engagement member is proximal of the distal hinge and distal of the proximal hinge.

12. The surgical ligation clip of claim 1, wherein the proximal hinge is defined by first and second walls spaced apart from each other by a slot.

13. The surgical ligation clip of claim 12, wherein the first and second walls have different thicknesses from each other.

14. The surgical ligation clip of claim 1, wherein the first and second arms include first and second protrusions, respectively, configured to be gripped by corresponding jaws of a robotic applicator.

15. The surgical ligation clip of claim 1, wherein each of the first and second arms includes a bioabsorbable material.

16. A surgical ligation clip comprising:
(a) a first arm extending from a proximal end to a first distal end, wherein the first arm includes a distal hook at the first distal end, wherein the distal hook includes first and second distal hook portions spaced apart from each other by a gap; and
(b) a second arm extending from the proximal end to a second distal end, wherein the second arm is pivotable relative to the first arm about a proximal hinge to clamp tissue between the first and second arms, wherein the second arm includes first and second distal legs pivotable relative to each other about a distal hinge.

17. The surgical ligation clip of claim 16, wherein the second arm is configured to be pivoted away from the first arm about the proximal hinge to define an open state of the surgical ligation clip, and wherein the second arm is configured to be pivoted toward the first arm about the proximal hinge to define a fully closed state of the surgical ligation clip.

18. The surgical ligation clip of claim 17, wherein the first and second distal hook portions are configured to cammingly engage the first and second distal legs, respectively, to pivot the first and second distal legs toward each other about the distal hinge for allowing the first and second distal legs to pass through the gap when the surgical ligation clip is transitioned from the open state to the fully closed state, wherein the first and second distal hook portions are configured to abut the first and second distal legs, respectively, to inhibit the surgical ligation clip from transitioning away from the fully closed state.

19. A surgical ligation clip comprising:
(a) a first arm extending from a first proximal end to a first distal end, wherein the first arm includes a distal hook at the first distal end;
(b) a second arm extending from a second proximal end to a second distal end, wherein the second arm includes a distal hook at the second distal end, wherein the second arm is movable relative to the first arm to clamp tissue between the first and second arms;
(c) a column extending between the first and second arms, wherein the column includes first and second column portions pivotable relative to each other about a hinge; and
(d) an arcuate hinge extending between the first and second arms proximally of the column.

20. The surgical ligation clip of claim 19, wherein the distal hook includes a pair of sidewalls configured to laterally flank the second distal end of the second arm when the surgical ligation clip is in a fully closed state to inhibit lateral misalignment of the first and second arms.
